(19) United States
(12) Patent Application Publication
(10) Pub. No.: US 2020/0111573 A1
(43) Pub. Date: Apr. 9, 2020

(54) SYSTEMS AND METHODS FOR AUTONOMOUS MACHINE INTERPRETATION OF HIGH THROUGHPUT BIOLOGICAL ASSAYS FOR EMBRYO SELECTION

(71) Applicant: CooperGenomics, Inc., Trumbull, CT (US)

(72) Inventors: John BURKE, Reno, NV (US); Michael J. LARGE, Spring, TX (US); Joshua BLAZEK, Houston, TX (US)

(21) Appl. No.: 16/595,384

(22) Filed: Oct. 7, 2019

Related U.S. Application Data
(60) Provisional application No. 62/742,211, filed on Oct. 5, 2018.

(51) Int. Cl. G16H 50/20; G16H 50/30; G16B 20/20; G16B 30/10; G16B 40/10; G16B 20/10; G06F 17/18; C12Q 1/6883; G16B 45/00; G06N 7/00; G06N 7/04

(52) U.S. Cl. CPC G16H 50/20; G16H 50/30; G16B 20/20; G16B 30/10; G16B 40/10; G06N 7/046; G06F 17/18; C12Q 1/6883; G16B 45/00; G06N 7/005; G16B 20/10

(57) ABSTRACT

A method for identifying chromosomal abnormalities in an embryo, is disclosed. Sample genomic sequence information obtained from an embryo is received, wherein the sample genomic sequence information is comprised of a plurality of genomic sequence reads. The sample genomic sequence information is aligned against a reference genome. The sample genomic sequence information is normalized against baseline genomic sequence information to correct the sample genomic sequence information for locus effects and generate a normalized sample genomic sequence information dataset. One or more correction factors derived from a regression analysis of error factors is applied to the normalized sample genomic sequence information dataset to correct for technical effects and generate de-noised sample genomic sequence information dataset. Copy number variations in the de-noised sample genomic sequence information dataset is identified when a frequency of genomic sequence reads aligned to a chromosomal position on the reference genome deviates from a frequency threshold.

### CROSS-REFERENCE TO RELATED APPLICATIONS

This application claims the benefit of priority under 35 U.S.C. § 119 from U.S. Provisional Patent Application 62/742,211 filed Oct. 5, 2018, which is incorporated herein by reference in its entirety

### FIELD

The embodiments disclosed herein are generally directed towards systems and methods for identifying embryo candidates for implantation into a womb. More specifically, there is a need for autonomous systems and methods for identifying chromosomal abnormalities in in vitro fertilized embryo candidates for implantation into a prospective mother.

### BACKGROUND

In vitro fertilization is intended to be followed by the implantation of an embryo into a prospective mother. Given an embryo, it is important to check for defects that may preclude the successful birth of a healthy child and given multiple embryos an optimal embryo must be chosen for each cycle of IVF to increase the probability of successful implantation.

In the past, microscopic inspection of embryo morphology or microscopic inspection of chromosome banding patterns was by used by clinical specialists to identify non-optimal embryos. These methods were sub optimal in resolution and inconsistent due to their reliance upon human operators. Conventional karyotyping is limited to detecting features greater than 5 mega-bases (mb) and FISH assays are limited to just under 1 mb and both are limited by a set of probes which must be designed for specific genomic loci. The use of human specialists to examine embryo candidates via microscopy introduces clerical and inspection error rates and other uncertainty into the embryo screening process.

The availability of next generation sequencing (NGS) provides whole genome coverage that requires much less custom design work than conventional karyotyping methods. Furthermore, assay cost can be controlled via sequencing depth which can also be optimized for a desired resolution where deeper sequencing allows for finer resolution.

But NGS karyotyping does have issues with respect to signal to noise. Specifically, due to confounding factors like sample handling, amplification bias, guanine-cytosine (GC) content and technical differences between different genomic loci; similarly sized regions of identical copy number will usually have very different sequence counts. The differences caused by these confounding factors are often greater in amplitude than differences caused by true changes in copy number. Therefore, accurate interpretation of NGS data requires methods that can effectively separate copy number signal from noise derived from confounding factors.

Moreover, given a de-noised copy number signal, interpretation into a cytogenetic status (calling aneuploids or segmental duplications/deletions) or a karyogram can also pose some challenges. The first issue is the volume of samples that must be processed by a laboratory. Another issue is the rate of artifacts (even in de noised data) that appear to be copy number variation features in genomic regions that are actually normal (normal=meaning somatic regions have copy number of 2, sex chromosome to 2 with at least 1 copy number belonging to Chr X). Also, not every copy number change is equal in clinical significance and chromosomal anomalies with serious consequences should be given more importance. Finally, previous and current methods are over reliant upon human inspection of plots which introduces uncertainty, error from subjectivity, fatigue, inadequate training, and other causes of inaccuracy.

As such, there is a need for methods or systems that can accurately/robustly identify chromosomal abnormalities in embryo candidates to allow for the selection of embryos that have the greatest chance of resulting in a successful pregnancy when implanted.

### SUMMARY

In one aspect, a method for identifying chromosomal abnormalities in an embryo, is disclosed. Sample genomic sequence information obtained from an embryo is received, wherein the sample genomic sequence information is comprised of a plurality of genomic sequence reads. The sample genomic sequence information is aligned against a reference genome. The sample genomic sequence information is normalized against baseline genomic sequence information to correct the sample genomic sequence information for locus effects and generate a normalized sample genomic sequence information dataset. One or more correction factors derived from a regression analysis of error factors is applied to the normalized sample genomic sequence information dataset to correct for technical effects and generate de-noised sample genomic sequence information dataset. Copy number variations in the de-noised sample genomic sequence information dataset is identified when a frequency of genomic sequence reads aligned to a chromosomal position on the reference genome deviates from a frequency threshold.

In another aspect, a system for identifying chromosomal abnormalities in an embryo, is disclosed. The system is comprised of a data store unit, a computing device and a display, which are all communicatively connected to each other.

The data store unit is configured to store sample genomic sequence information obtained from an embryo. The computing device hosts a data de-noising engine and an interpretation engine. The data de-noising engine is configured to receive the sample genomic sequence information from the data store, normalize the sample genomic sequence information against baseline genomic sequence information to correct the sample genomic sequence information for locus effects, and apply one or more correction factors derived from a regression analysis of error factors to correct for technical effects and generate de-noised sample genomic sequence information dataset. The interpretation engine is configured to identify copy number variations in the de-noised sample genomic sequence information dataset when a frequency of genomic sequence reads aligned to a chromosomal position in the de-noised sample genomic sequence information dataset deviates from a frequency threshold.

The display is configured to display a report containing the identified copy number variations.

In still another aspect, a method for identifying sex aneuploidy in an embryo, is disclosed. Sample genomic sequence information obtained from an embryo is received, wherein the sample genomic sequence information is comprised of a plurality of genomic sequence reads. The sample genomic sequence information is aligned against a reference genome. The sample genomic sequence information is normalized against baseline genomic sequence information to correct the sample genomic sequence information for locus effects and generate a normalized sample genomic sequence information dataset. One or more correction factors derived from a regression analysis of error factors is applied to the normalized sample genomic sequence information dataset to correct for technical effects and generate a de-noised sample genomic sequence information dataset. A trained neural network is utilized to analyze the de-noised sample genomic sequence information dataset and classify the sex aneuploidy status of the embryo.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIGS. 1A-1E are BLUEFUSE® visualization graphs that depict embryos with normal and abnormal chromosomal conditions, in accordance with various embodiments.

FIG. 2 is an exemplary flowchart showing a method for identifying chromosomal abnormalities, in accordance with various embodiments.

FIG. 3 illustrates how read counts are normalized for locus effects, in accordance with various embodiments.

FIG. 4 is a plot that illustrates an evaluation of the similarities between samples of interest and baseline samples, in accordance with various embodiments.

FIG. 5 is a depiction of how to construct a baseline vector from multiple baseline samples in a baseline set, in accordance with various embodiments.

FIG. 6A is a plot that illustrates bin effect normalization of embryo data, in accordance with various embodiments.

FIG. 6B is a plot that illustrates real-time sample effect corrections, in accordance with various embodiments.

FIG. 7 is a depiction of how LOWESS techniques can be used for GC correction, in accordance with various embodiments.

FIGS. 8A-8B are plots that show GC technical effect on bin score, in accordance with various embodiments.

FIG. 9 is a schematic diagram of a system for identifying chromosomal abnormalities in an embryo, in accordance with various embodiments.

FIG. 10 is a block diagram that illustrates a computer system, in accordance with various embodiments.

FIG. 11 is an exemplary flowchart showing a method for identifying sex aneuploidy in an embryo, in accordance with various embodiments.

FIG. 12 is a depiction of a Hidden Markov Model (HMM) finite state machine topology, in accordance with various embodiments.

FIGS. 13A-B are de-noised and normalized plots that show a deletion at chromosome 15, in accordance with various embodiments.

FIG. 14 is a plot that depicts a method that uses chromosomal clusters to determine complex embryo sex aneuploidy, in accordance with various embodiments.

FIG. 15 is a depiction of a normalized and de-noised bin data neural network for the prediction of complex sex aneuploidy in an embryo, in accordance with various embodiments.

FIG. 16 is a depiction of a feed forward network structure, in accordance with various embodiments.

FIG. 17 is a graph showing the net change in the various ploidy classifications when comparing the improved systems and methods disclosed herein (PGTai) against the conventional subjective calling methods (BLUEFUSE® software offered by ILLUMINA®), in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

This specification describes various exemplary embodiments of systems and methods for identifying chromosomal abnormalities in in vitro fertilized embryo candidates for implantation. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. The Human reference genome is a representation of one of these strands (which as used herein, is called strand 1). As used herein, the reverse compliment of strand 1 is called strand 2. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The phrase “next generation sequencing” (NGS) refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the MISEQ, HISEQ and NEXTSEQ Systems of Illumina and the Personal Genome Machine (PGM) and SOLiD Sequencing System of Life Technologies Corp, provide massively parallel sequencing of whole or targeted genomes. The SOLiD System and associated workflows, protocols, chemistries, etc. are described in more detail in PCT Publication No. WO 2006/084132, entitled “Reagents, Methods, and Libraries for Bead-Based Sequencing,” international filing date Feb. 1, 2006, U.S. patent application Ser. No. 12/873,190, entitled “Low-Volume Sequencing System and Method of Use,” filed on Aug. 31, 2010, and U.S. patent application Ser. No. 12/873,132, entitled “Fast-Indexing Filter Wheel and Method of Use,” filed on Aug. 31, 2010, the entirety of each of these applications being incorporated herein by reference thereto.

The phrase “sequencing run” refers to any step or portion of a sequencing experiment performed to determine some information relating to at least one biomolecule (e.g., nucleic acid molecule).

As used herein, the phrase “genomic features” can refer to a genome region with some annotated function (e.g., a gene, protein coding sequence, mRNA, tRNA, rRNA, repeat sequence, inverted repeat, miRNA, siRNA, etc.) or a genetic/genomic variant (e.g., single nucleotide polymorphism/variant, insertion/deletion sequence, copy number variation, inversion, etc.) which denotes a single or a grouping of genes (in DNA or RNA) that have undergone changes as referenced against a particular species or sub-populations within a particular species due to mutations, recombination/crossover or genetic drift.

Genomic variants can be identified using a variety of techniques, including, but not limited to: array-based methods (e.g., DNA microarrays, etc.), real-time/digital/quantitative PCR instrument methods and whole or targeted nucleic acid sequencing systems (e.g., NGS systems, Capillary Electrophoresis systems, etc.). With nucleic acid sequencing, coverage data can be available at single base resolution.

The phrase “fragment library” refers to a collection of nucleic acid fragments, wherein one or more fragments are used as a sequencing template. A fragment library can be generated, for example, by cutting or shearing a larger nucleic acid into smaller fragments. Fragment libraries can be generated from naturally occurring nucleic acids, such as mammalian or bacterial nucleic acids. Libraries comprising similarly sized synthetic nucleic acid sequences can also be generated to create a synthetic fragment library.

The phrase “chromosomal abnormality” or “chromosomal abnormalities” denotes both structural (e.g., deletions, duplications, translocations, inversions, insertions, etc.) and numerical (i.e., aneuploidy) chromosomal disorders.

The phrase “mosaic embryo” denotes embryos containing two or more cytogentically distinct cell lines. For example, a mosaic embryo can contain cell lines with different types of aneuploidy or a mixture of euploid and genetically abnormal cells containing DNA with genetic variants that may be deleterious to the viability of the embryo during pregnancy.

In various embodiments, a sequence alignment method can align a fragment sequence to a reference sequence or another fragment sequence. The fragment sequence can be obtained from a fragment library, a paired-end library, a mate-pair library, a concatenated fragment library, or another type of library that may be reflected or represented by nucleic acid sequence information including for example, RNA, DNA, and protein based sequence information. Generally, the length of the fragment sequence can be substantially less than the length of the reference sequence. The fragment sequence and the reference sequence can each include a sequence of symbols. The alignment of the fragment sequence and the reference sequence can include a limited number of mismatches between the symbols of the fragment sequence and the symbols of the reference sequence. Generally, the fragment sequence can be aligned to a portion of the reference sequence in order to minimize the number of mismatches between the fragment sequence and the reference sequence.

In particular embodiments, the symbols of the fragment sequence and the reference sequence can represent the composition of biomolecules. For example, the symbols can correspond to identity of nucleotides in a nucleic acid, such as RNA or DNA, or the identity of amino acids in a protein. In some embodiments, the symbols can have a direct correlation to these subcomponents of the biomolecules. For example, each symbol can represent a single base of a polynucleotide. In other embodiments, each symbol can represent two or more adjacent subcomponent of the biomolecules, such as two adjacent bases of a polynucleotide. Additionally, the symbols can represent overlapping sets of adjacent subcomponents or distinct sets of adjacent subcomponents. For example, when each symbol represents two adjacent bases of a polynucleotide, two adjacent symbols representing overlapping sets can correspond to three bases of polynucleotide sequence, whereas two adjacent symbols representing distinct sets can represent a sequence of four bases. Further, the symbols can correspond directly to the subcomponents, such as nucleotides, or they can correspond to a color call or other indirect measure of the subcomponents. For example, the symbols can correspond to an incorporation or non-incorporation for a particular nucleotide flow.

In various embodiments, a computer program product can include instructions to select a contiguous portion of a fragment sequence; instructions to map the contiguous portion of the fragment sequence to a reference sequence using an approximate string matching method that produces at least one match of the contiguous portion to the reference sequence.

In various embodiments, a system for nucleic acid sequence analysis can include a data analysis unit. The data analysis unit can be configured to obtain a fragment sequence from a sequencing instrument, obtain a reference sequence, select a contiguous portion of the fragment sequence, and map the contiguous portion of the fragment sequence to the reference sequence using an approximate string mapping method that produces at least one match of the contiguous potion to the reference sequence.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, the term “cell” is used interchangeably with the term “biological cell.” Non-limiting examples of biological cells include eukaryotic cells, plant cells, animal cells, such as mammalian cells, reptilian cells, avian cells, fish cells, or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells, and the like. A mammalian cell can be, for example, from a human, a mouse, a rat, a horse, a goat, a sheep, a cow, a primate, or the like

### Conventional Methods for Processing NGS Data to Identify Chromosomal Abnormalities

Many clinical pipelines that use NGS data follow similar initial workflows. First, the raw sequences generated using a sequencing machine are demultiplexed; when many samples are sequenced simultaneously, sequences from different subjects are tagged with initial barcodes which are removed after a sequence is assigned to a subject. Adapters or other artificial features are removed from the generated sequences. Sequences are often assigned to genomic loci by computer programs that align or match the bases of the generated sequence to a known genomic reference sequence and PCR duplicates and low-quality sequences are often removed during or shortly after the alignment process. Sequences that have been processed and matched to a locus are often called aligned sequences or aligned reads. The number of sequences generated from each sample of interest is often called the “sequencing depth”.

A commercial implementation of a conventional approach to copy number variation (CNV) calling is provided by Illumina (BLUEFUSE®) which also smooths data by taking medians within a sliding window over k proximal bins.

CNVs are genomic alterations that result in an abnormal number of copies of one or more genes and can contribute to diseases. BLUEFUSE® software generates a graph that allows users to visualize, analyze, and interpret for genetic abnormalities.

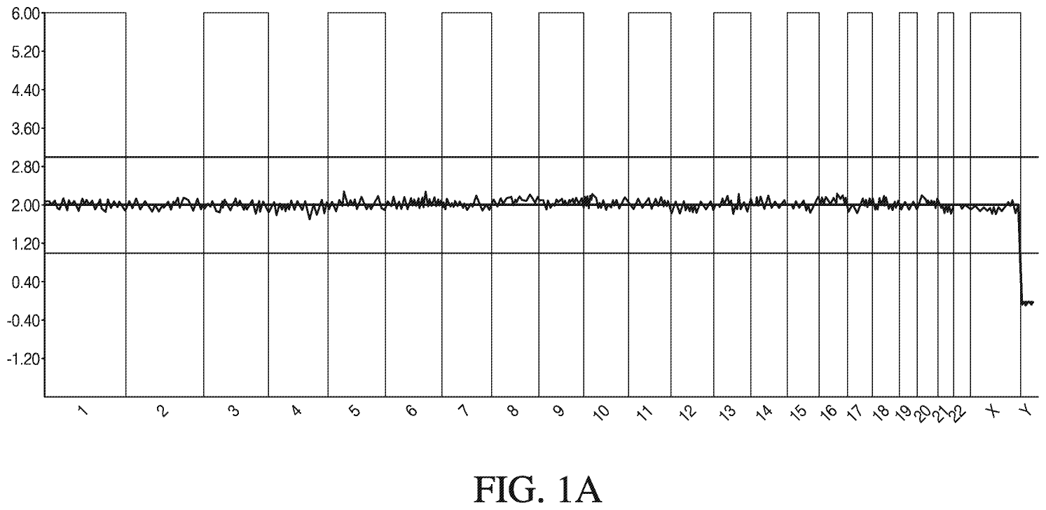

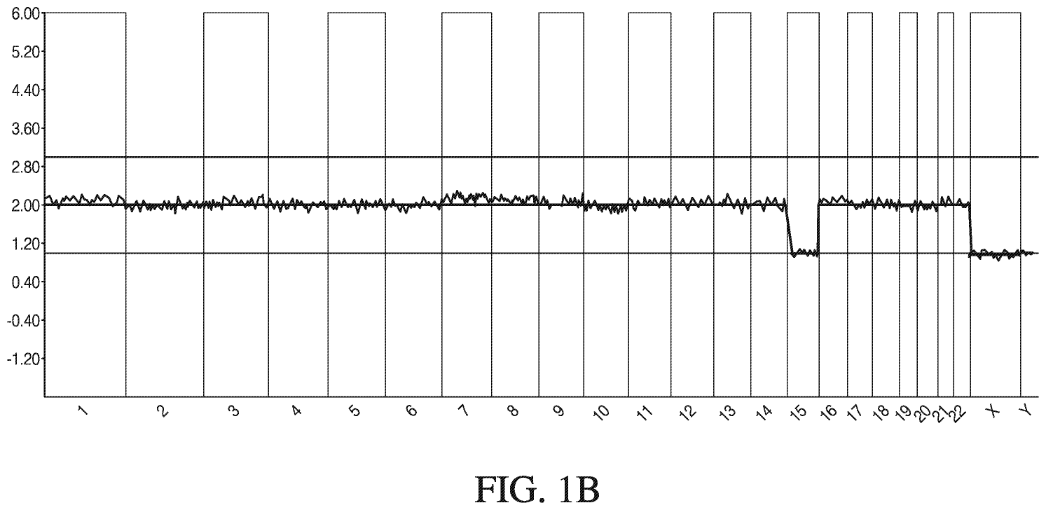

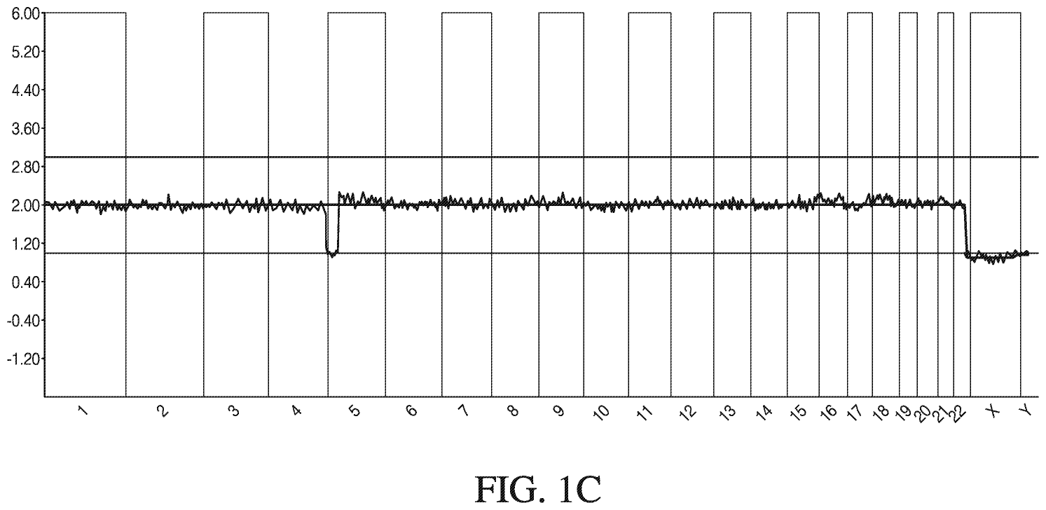

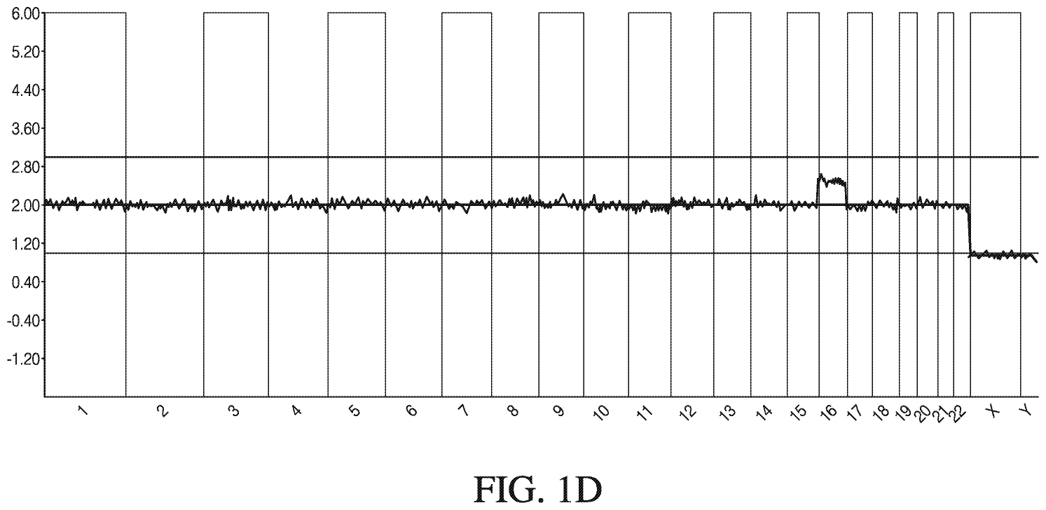

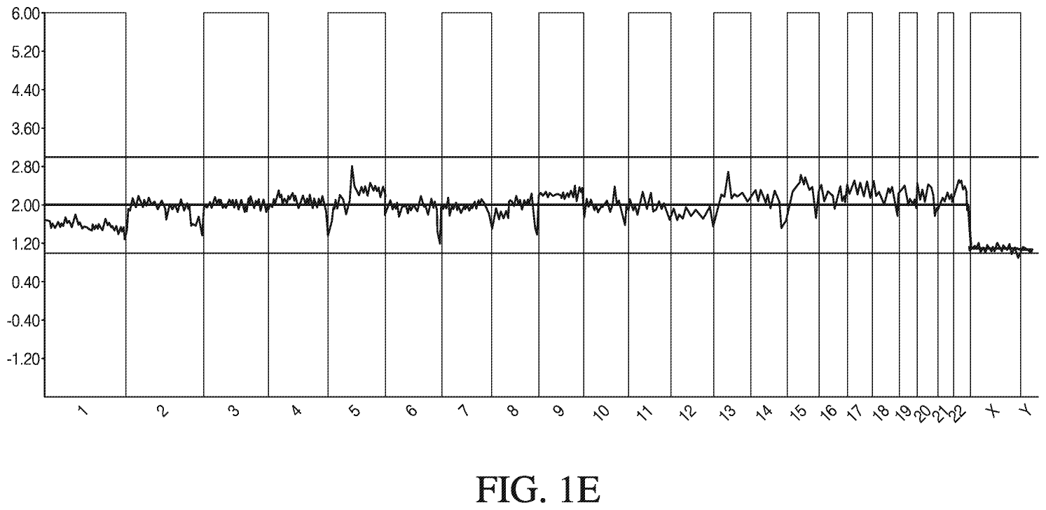

An embryo with a normal number of chromosomes is a Euploid embryo. As depicted in FIG. 1A, the euploid embryo is visualized on the BLUEFUSE® graph as having two copies (on the y-axis of the graph) of each chromosome number (1-22) shown on the x-axis of the graph. In terms of sex, female embryos have two copies of the X chromosome and no copies of the Y chromosome (as depicted in FIG. 1A), and male embryos have one copy of the X chromosome and one copy of the Y chromosome.

An embryo with an abnormal number of chromosomes, on the other hand, is an Aneuploid embryo. A chromosome with a copy gain (three copies instead of the normal two copies) is called trisomy, and a chromosome with a copy loss (one copy instead of the normal two copies) is called monosomy. FIG. 1B depicts a male aneuploid embryo with monosomy. Two copies are visualized for chromosomes 1-14, 16-22, and only one copy of chromosome 15 (monosomy). There is also one copy of chromosome X and chromosome Y which indicates that the embryo is male.

When only part of a chromosome is copied or deleted abnormally, it is called a duplication or deletion, respectively. FIG. 1C depicts a male embryo with a deletion on chromosome 5. Two copies are visualized for chromosomes 1-4, 6-22 and part of chromosome 5 is deleted. There is also one copy of chromosome X and chromosome Y which indicates that the embryo is male.

An embryo which possesses both normal and abnormal cells for a particular chromosome is called a Mosaic embryo. Visually, this embryo has a chromosomal copy number that is in between normal (two copies) and abnormal (either one copy or three copies, depending on if it is Trisomy or Monosomy). FIG. 1D depicts a male embryo with a mosaic chromosome 16. Two copies are visualized for chromosomes 1-15, 17-22, and chromosome 16 is mosaic (with a copy number of 2.5). There is also one copy of chromosome X and chromosome Y which indicates that the embryo is male.

There are significant limitations to the approach taken by the BLUEFUSE® software. If the quality of the embryo biopsy has been compromised, the DNA has degraded, or if there are issues with the library preparation itself, it becomes more difficult to interpret the data, as the noise (background) level of the data increases. Higher noise levels make it challenging to decipher which changes from normal may be real genetic abnormalities versus issues with the DNA quality itself. The result of these shortcomings is that segmental or mosaic calls, or complex sex aneuploidy calls must be made by a human technician by inspection of plots of the normalized bin scores. The subjectivity and uncertainty associated with human interpretation of the images can lead to unwanted variations in the analysis of the embryos for chromosomal abnormalities. FIG. 1E depicts a male embryo with high noise levels, making it difficult for a human technician to interpret whether there are true genetic abnormalities in the embryo.

### Automated Machine Interpretation Methods for Processing NGS Data to Identify Chromosomal Abnormalities

Systems and methods for automated detection of chromosomal abnormalities including segmental duplications/deletions, mosaic features, as well as complex sex aneuploidy, are disclosed. Conceptually, these systems and methods have two primary pipelines: 1) de-noising/normalization (to de-noise the raw sequence reads), and 2) interpretation (to decode the de-noised/normalized signals into karyograms and clinical aneuploidy calls).

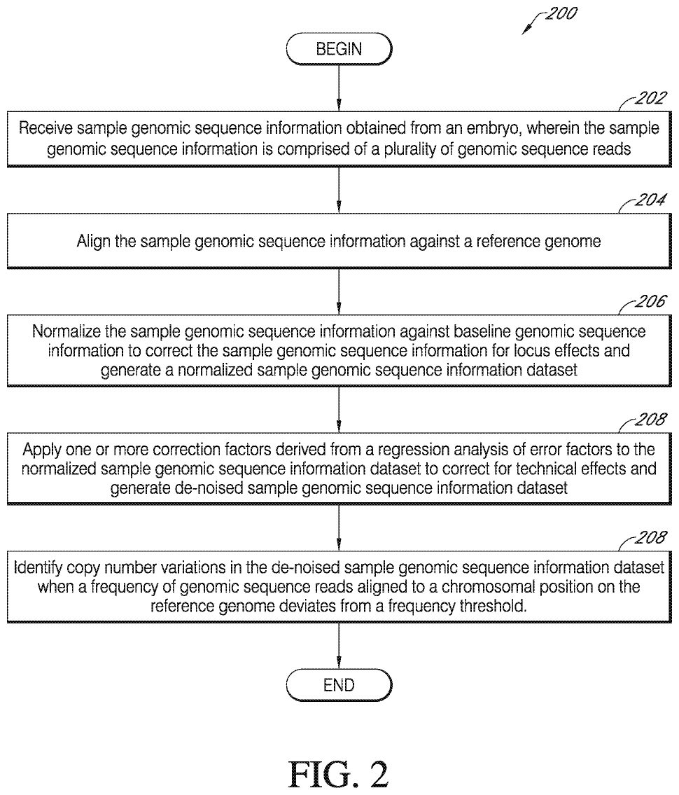

FIG. 2 is an exemplary flowchart showing a method 200 for automated identification of chromosomal abnormalities in an embryo, in accordance with various embodiments. In step 202, sample genomic sequence information obtained from an embryo is received. The sample genomic information is comprised of a plurality of genomic sequence reads generated using various genomic sequencing techniques including NGS, PCR, etc. In step 204, the sample genomic sequence information is aligned against a reference genome. In various embodiments, the reference genome is a human reference genome.

In step 206, the sample genomic sequence information is normalized against baseline genomic sequence information to correct the sample genomic sequence information for locus effects. Locus effects are aspects of a genomic location that are associated with a change in sequence coverage even when is no change in copy number. Examples of locus effects can be, but are not limited to: 1) GC content within 50, 100, 150, etc. . . . bases of a base position, 2) potential for the DNA around a genomic location to form secondary structures, 3) sequence similarity to other genomic locations, etc.

In various embodiments, normalizing the sample genomic sequence information for locus effects involves first setting a bin size. In various embodiments, the bin size is set to 1 megabase (mb). It should be understood, however, that the bin size can be set to any size, including: 100 kb, 500 kb, or any other value between 1 million and to 20 million as long as it doesn't exceed the length of the human genome. Next, the sample genomic sequence information and baseline genomic sequence information is segmented into a plurality of bins based on the bin size. Then, the number of genomic sequence reads from the sample genomic sequence information that is aligned to each of the plurality of sample genomic sequence information bins is determined to generate sample bin scores for each of the plurality of sample genomic sequence information bins.

Next, the number of genomic sequence reads from the baseline genomic sequence information that is aligned to each of the plurality of baseline genomic sequence information bins is determined to generate baseline bin scores for each of the plurality of baseline genomic sequence information bins. Then, the sample bin scores are normalized against the baseline bin scores to generate a normalized sample genomic sequence dataset.

In various embodiments, the baseline bin scores were determined by first receiving a plurality of baseline genomic sequence information datasets obtained from euploid embryos. The bin scores for each of the plurality of baseline genomic sequence information datasets were then determined. Next, a subset of baseline genomic sequence information datasets with bin scores that exceed a similarity threshold to the sample genomic sequence information were selected from the plurality of baseline genomic sequence information datasets. Finally, the baseline bin scores were generated by determining the median values of bin scores in the selected subset of baseline genomic information datasets.

In step 208, one or more correction factors derived from a regression analysis of error factors was applied to correct for technical effects and generate a de-noised sample genomic sequence information dataset.

In step 210, CNVs are identified from the de-noised sample genomic sequence information dataset when a frequency of genomic sequence reads aligned to a chromosomal position on the reference genome deviates from a frequency threshold.

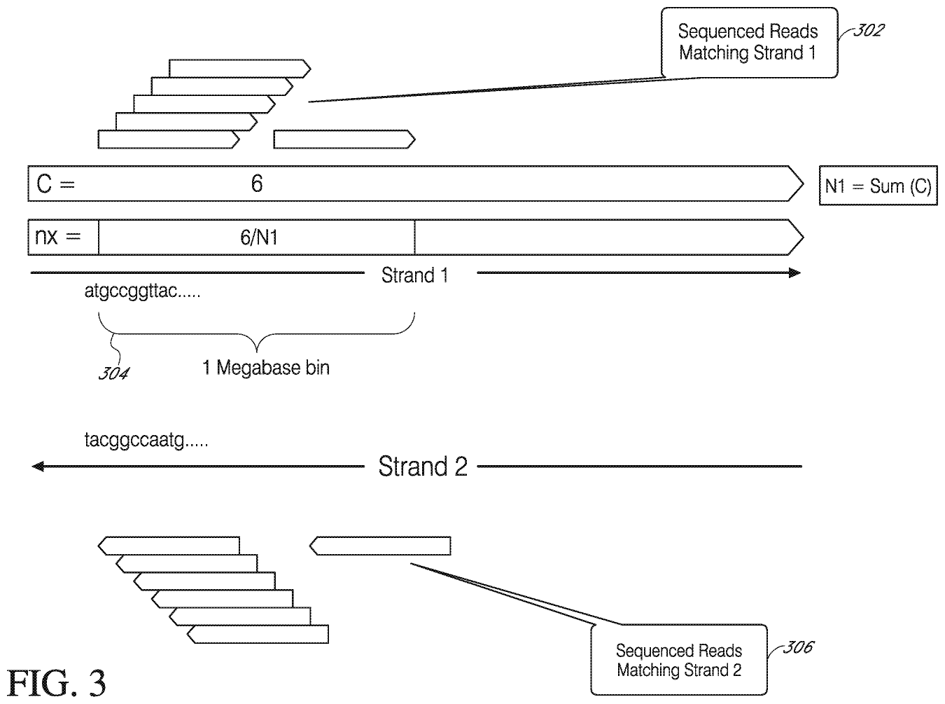

Various aspects of method 200 are shown in FIGS. 3-8B. As shown in FIG. 3, for each strand (strand 1 and strand 2 of the Human genome as described above) and for each bin, nx is defined as the bin count scaled by the total number of reads 302 aligned to diploid chromosomes for the sample of interest on the same strand.

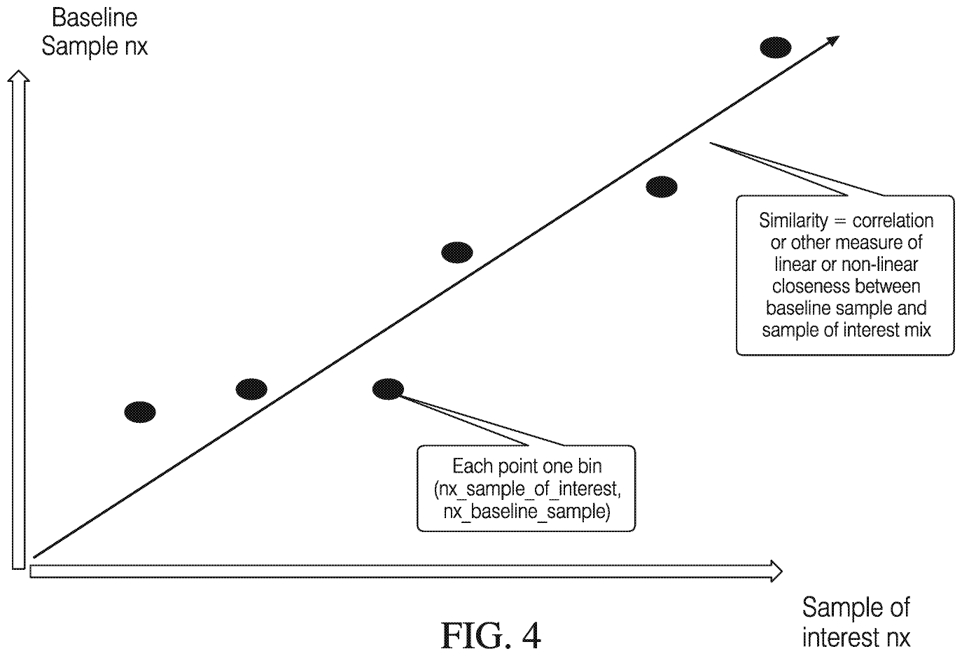

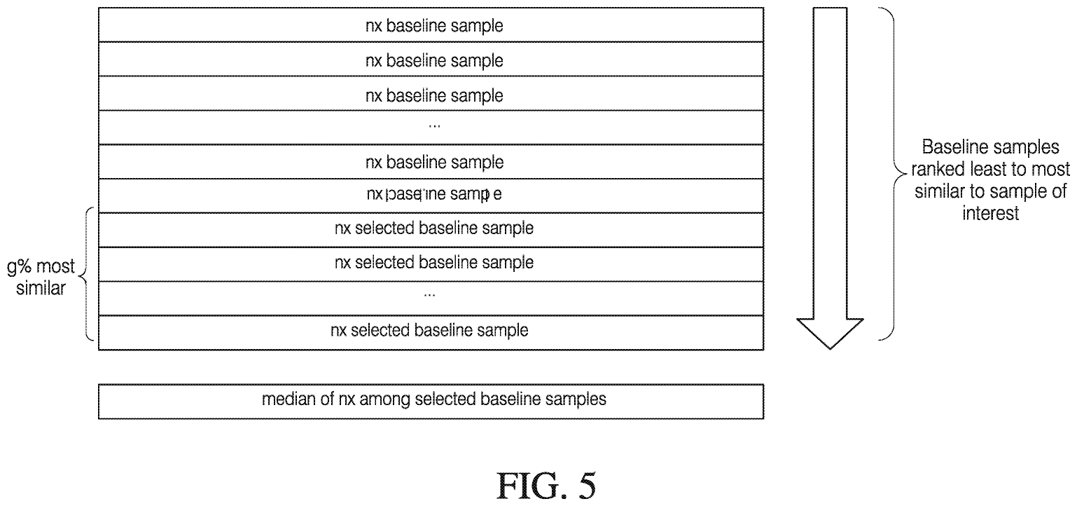

As shown in FIG. 4, the first correction for locus (bin) effects can be done by normalizing bin counts from the sample of interest against a baseline set of euploid samples. The bin size can be first set to 1 megabase 304. It should be appreciated, however, that bin size can be set to any size essentially, including: 100 kb, 500 kb, or any other value between 1 and 20 million. Next, as shown in FIG. 5, the sample genomic sequence information is segmented into a plurality of bins and an optimal subset of baseline samples is then selected (instead of using the entire baseline set) to be normalize for bin effects where optimality is defined as having baseline nx most similar to the sample of interest nx. Similarity is then quantified as the correlation of nx for a baseline sample and nx for the sample of interest. In various embodiments, rank correlation can also be used as a measure of similarity although there are many alternatives (such as MSE/residual sum squares, Euclidian distance or Mahalanobis distance).

Given the above methods for calculating similarity between the sample of interest and a baseline sample, samples from the baseline with highest similarity to the sample of interest were selected.

Given the set of similarity values s={s1, s2, . . . , s (number baseline samples)}, the similarity between baseline samples and the sample of interest, baseline samples with s>t were selected where t is the gth percentile of s. In various embodiments, the parameter g can be set to 90% but can also be set to 10%, 30%, 50%, 80% or any other number between 1 and 100. In addition to correcting bin marginal effects on locus counts, this corrects for distal bins with correlated scores where the coverage of one bin informs the coverage of another bin. After an optimal sub-set of baseline samples are selected, the sample of interest's bin scores are normalized by the median baseline-subset normalized bin scores. Normalization can then be done by division and the result is a vector of bin scores centered at 1.0.

One benefit of these methods for correcting for locus effects is that run samples are accumulated and euploid samples inform future normalization thus making normalized bin scores less noisy and the over system more accurate over time.

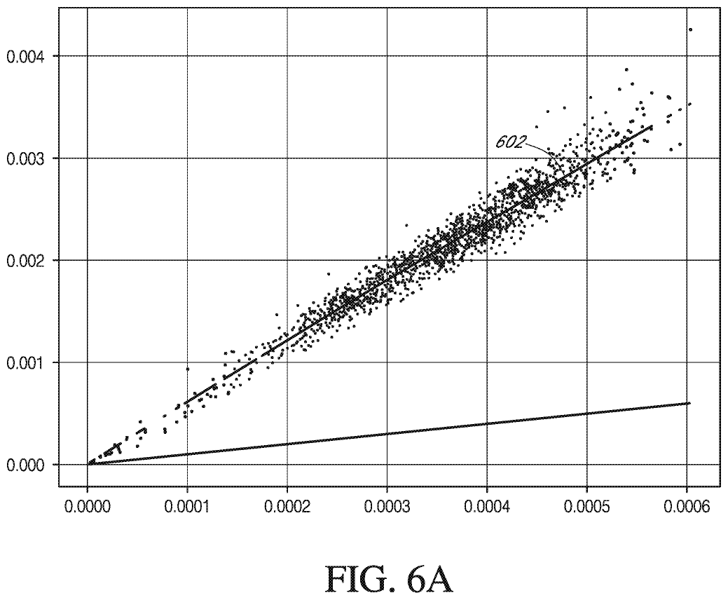

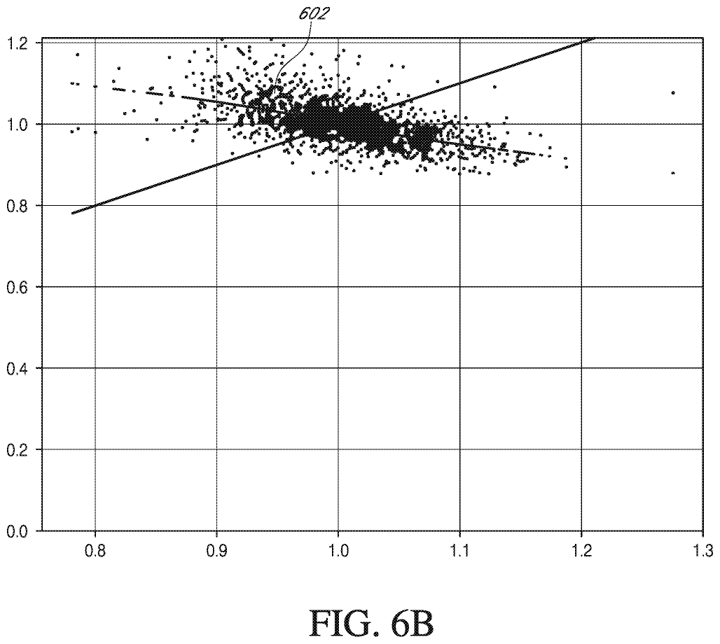

Biological processes specific to the state of the sample of interest at the time of sequencing (i.e., real-time sample effects), such as gene expression or regulation can also potentially affect genome availability during the sequencing process, but they can be corrected. One result of these real-time effects is signal attenuation of individual strands. Locally weighted Scatterplot Smoothing (LOWESS) estimators can be used to derive strand specific correction of bin signal by r=(the proportion of bin score from the forward strand). The strand specific bin score can then be normalized (divided) by this correction factor. As shown in FIGS. 6A and 6B, LOWESS calculates a correction factor 602 at each value of r by estimation of a low degree polynomial fit centered at r that only uses the sub-set of data points (r, bin_score) with values closest to r.

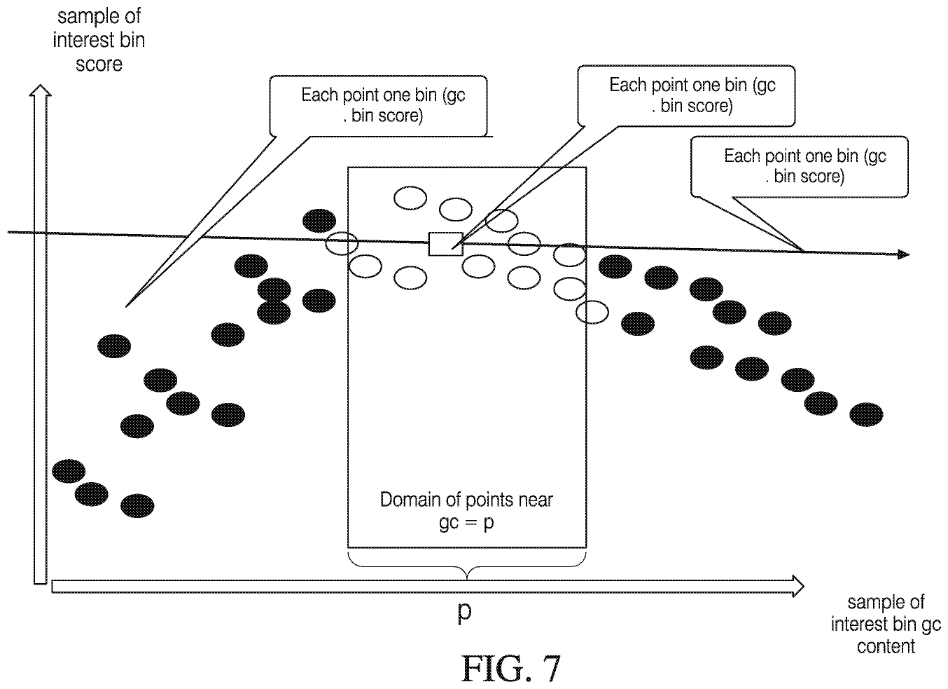

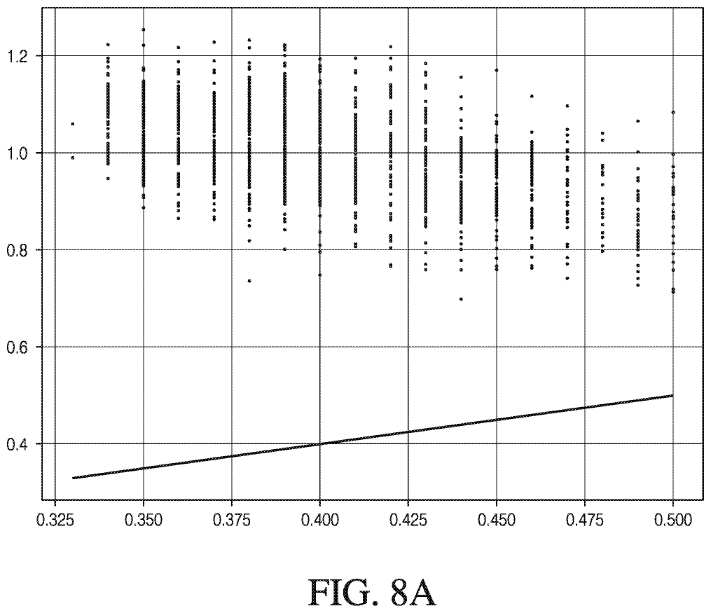

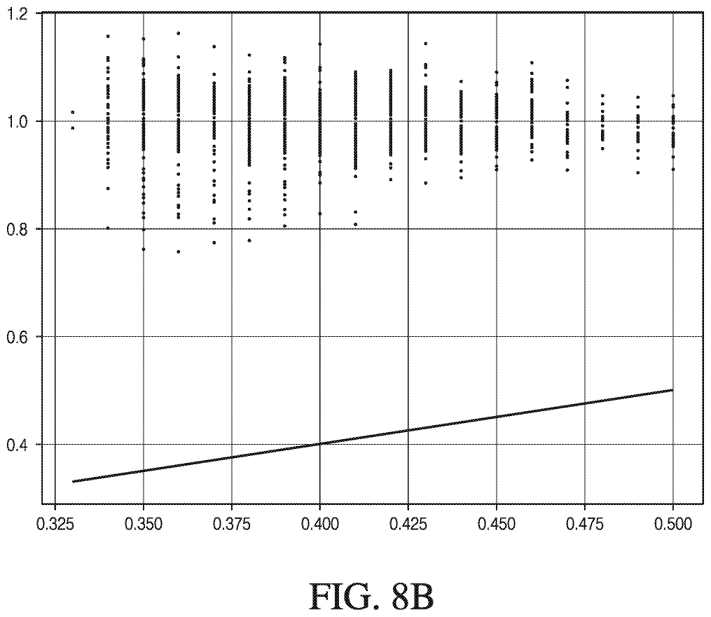

As noted above, the locus specific concentration of “c” and “g” bases and other technical effects (such as amplification bias, secondary structures, nucleosome density, miRNA interdiction, gene-expression, etc.) can affect sequence counts in bins; however, the above locus effects correction does not account for the differential response of each sample to these technical effects. There are many technical effects relevant for sample interaction correction. As shown in FIG. 7, GC content effects can be corrected for using LOWESS also. LOWESS can be used to define a correction for each level of the technical effects and normalize (subtract) the bin score by the factor. As shown in FIGS. 8A and 8B, LOWESS calculates a correction at each value, p, of gc percentage by estimation of a low degree polynomial fit centered at p that only uses the sub-set of data points (gc, bin_score) with gc values closest to p.

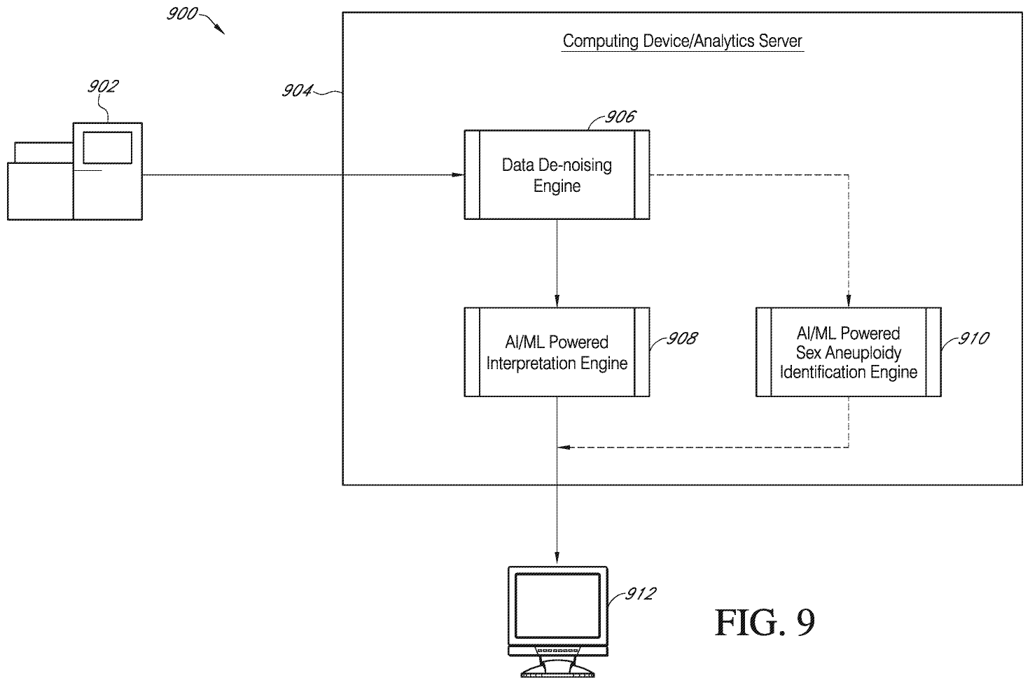

FIG. 9 is a schematic diagram of a system for identifying chromosomal abnormalities in an embryo, in accordance with various embodiments. The system 900 includes a sequencer 902, a computing device/analytics server 904 and a display 912.

The sequencer 902 is communicatively connected to the computing device/analytics server 904. In various embodiments, the computing device 904 can be communicatively connected to the genomic sequencer 902 via a network connection that can be either a “hardwired” physical network connection (e.g., Internet, LAN, WAN, VPN, etc.) or a wireless network connection (e.g., Wi-Fi, WLAN, etc.). In various embodiments, the computing device 904 can be a workstation, mainframe computer, distributed computing node (part of a “cloud computing” or distributed networking system), personal computer, mobile device, etc. In various embodiments, the genomic sequencer 902 can be a nucleic acid sequencer (e.g., NGS, Capillary Electrophoresis system, etc.), real-time/digital/quantitative PCR instrument, microarray scanner, etc. It should be understood, however, that the genomic sequencer 902 can essentially be any type of instrument that can generate nucleic acid sequence data from samples containing genomic fragments.

It will be appreciated by one skilled in the art that various embodiments of genomic sequencer 502 can be used to practice variety of sequencing methods including ligation-based methods, sequencing by synthesis, single molecule methods, nanopore sequencing, and other sequencing techniques. Ligation sequencing can include single ligation techniques, or change ligation techniques where multiple ligation are performed in sequence on a single primary nucleic acid sequence strand. Sequencing by synthesis can include the incorporation of dye labeled nucleotides, chain termination, ion/proton sequencing, pyrophosphate sequencing, or the like. Single molecule techniques can include continuous sequencing, where the identity of the nuclear type is determined during incorporation without the need to pause or delay the sequencing reaction, or staggered sequence, where the sequencing reactions is paused to determine the identity of the incorporated nucleotide.

In various embodiments, the genomic sequencer 902 can determine the sequence of a nucleic acid, such as a polynucleotide or an oligonucleotide. The nucleic acid can include DNA or RNA, and can be single stranded, such as ssDNA and RNA, or double stranded, such as dsDNA or a RNA/cDNA pair. In various embodiments, the nucleic acid can include or be derived from a fragment library, a mate pair library, a chromatin immuno-precipitation (ChIP) fragment, or the like. In particular embodiments, the genomic sequencer 902 can obtain the sequence information from a single nucleic acid molecule or from a group of substantially identical nucleic acid molecules.

In various embodiments, the genomic sequencer 902 can output nucleic acid sequencing read data (genomic sequence information) in a variety of different output data file types/formats, including, but not limited to: *.fasta, *.csfasta, *.xsq, *seq.txt, *qseq.txt, *.fastq, *.sff, *prb.txt, *.sms, *srs and/or *.qv.

In various embodiments, sequencer 902 further includes a data store configured to store sample genomic sequencing information that is generated by the sequencer 902 during a sample run.

The computing device/analytics sever 904 can be configured to host a Data De-Noising Engine 906, an Artificial Intelligence (AI)/Machine Learning (ML) Powered Interpretation Engine 908 and an AI/ML Powered Sex Aneuploidy Identification Engine 910.

The Data De-Noising Engine 906 can be configured to receive sample genomic sequence information from the sequencer 902 (or a data store associated with the sequencer 902), normalize the sample genomic sequence information against baseline genomic sequence information to correct the sample genomic sequence information for locus effects and apply one or more correction factors derived from a regression analysis of sampling error factors to correct for technical effects and generate a de-noised sample genomic sequence information dataset.

The AI/ML Powered Interpretation Engine 908 can be configured to identify copy number variations in the de-noised sample genomic sequence information dataset when a frequency of genomic sequence reads aligned to a chromosomal position in the de-noised sample genomic sequence information dataset deviates from a frequency threshold.

The AI/ML Powered Sex Aneuploidy Engine 910 can be configured to utilize a trained neural network to analyze the de-noised sample genomic sequence information dataset and classify the sex aneuploidy status of the embryo.

After the chromosomal abnormalities have been identified, the results can be displayed on a display or client terminal 912 that is communicatively connected to the computing device 904. In various embodiments, client terminal 912 can be a thin client computing device. In various embodiments, client terminal 912 can be a personal computing device having a web browser (e.g., INTERNET EXPLORER™, FIREFOX™, SAFARI™, etc) that can be used to control the operation of the Data De-Noising Engine 906, the Artificial Intelligence (AI)/Machine Learning (ML) Powered Interpretation Engine 908 and/or the AI/ML Powered Sex Aneuploidy Identification Engine 910.

### Interpretation

When bin-level normalization and de-noising is complete, bin-scores are centered at 1.0 (which represents copy number state 2). Machine learning and “artificial intelligence” methods can then be used to interpret (or decode) locus scores into Karyograms and clinical aneuploidy calls.

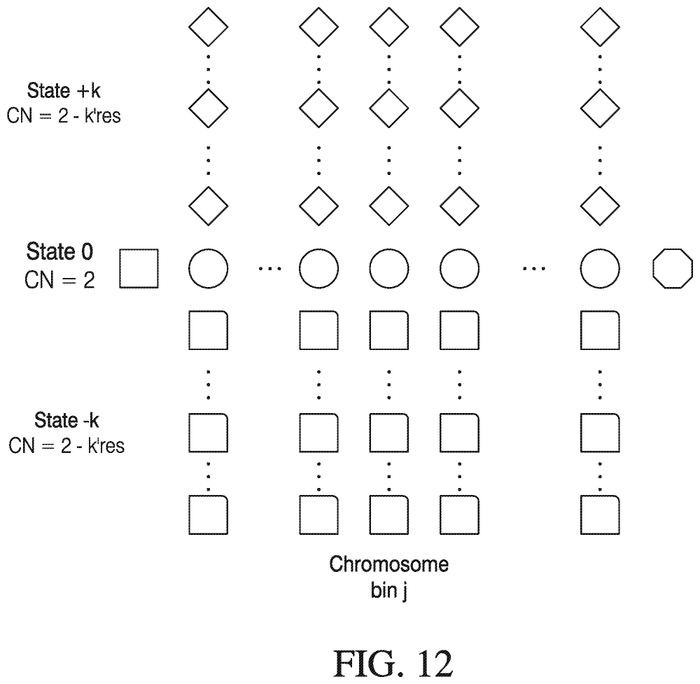

As shown in FIG. 12, Hidden Markov Models (HMMs) are a family of machine learning techniques common in speech recognition and signal processing. For each chromosome, a finite state machine is constructed with emission and transition probabilities parameterized by input data characteristics and the resolution desired by the user.

At each chromosome position, j, the model has a number of states, each state representing fraction of a copy number change. Initial states are all given equal probability and the transitions between states when advancing to the next genomic bin is defined by duration modeling that, on average, makes regions of >=3 megabases (this is a configurable parameter so that at megabase binsize the probability of remaining in a non 2.0 copy number state is ⅓ and all other transitions have equal probability). The scores emitted by each state follow a normal distribution (different distributions are possible in the scope of this invention) with standard deviation estimated from bin scores and mean value (k*res)/2.0 for a copy number value k*res where res is a defined resolution (by default 0.01). The process of assigning bins to a copy number given our HMM is called decoding which performed using a forward-backward algorithm which is a standard method of assigning a probability of membership in a state to each observation. Other decoding algorithms, like Viterbi, can also be used. The initial decoding by the forward backward algorithm defines the probability that each bin exists in each state, and thus, assigns each bin to a copy number state.

In various embodiments, the systems and methods disclosed herein can accommodate non-uniformity of the data. In the “Blue Fuse” methods described above, a constant variance (default 0.33) is assumed for all samples across all loci. As disclosed herein, the HMM is, by default, parameterized by the dynamically calculated variance of the sample of interest which allows more resolution for samples with lower variance (often samples with higher sequencing depth or DNA quality) and controls the number of false positive non-diploid assignments for more variable samples (often samples with lower sequencing depth or DNA quality).

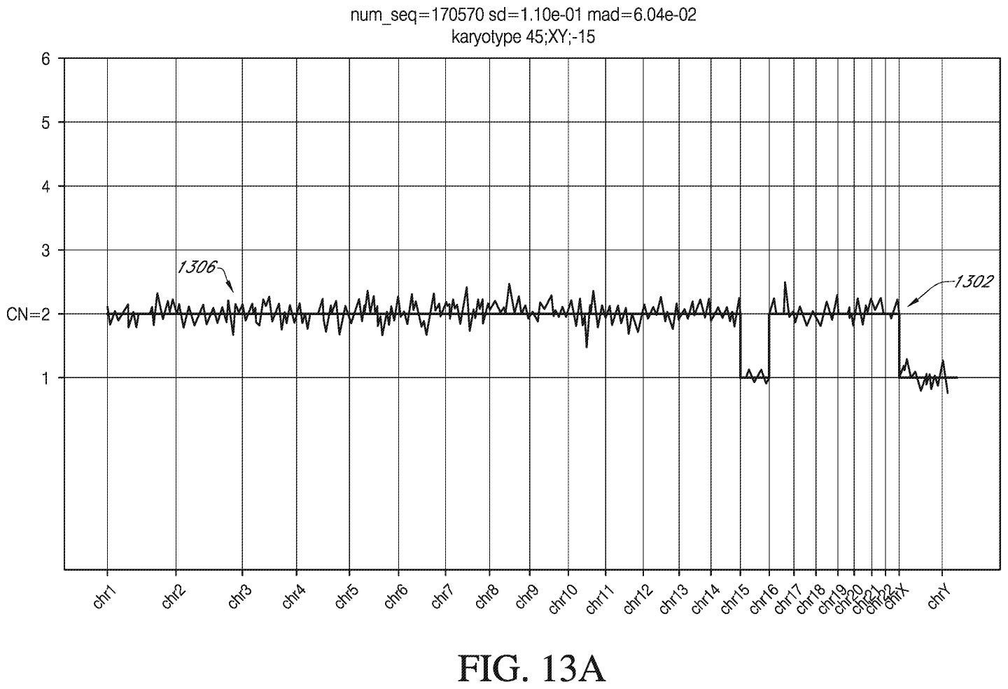

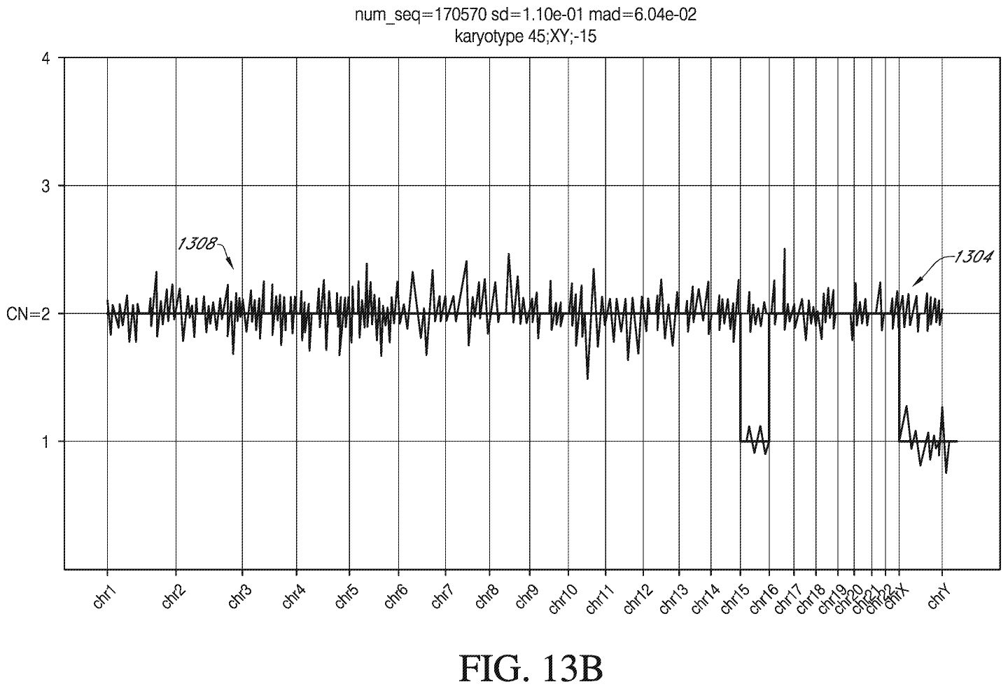

In various embodiments, the systems and methods disclosed herein uses machine learning to assign copy numbers to loci so that non-homogeneity and hetero-scedasticity in the data can be accounted for. For example, as shown in FIGS. 13A-13B, while normalized and de-noised bin scores have a constant center, they have different spreads or standard-deviations. In particular, FIG. 13A depicts a karyogram graph showing a deletion at chromosome 15. The de-noised and normalized bin scores 1306 are distributed more tightly around the decoded copy number line 1302. FIG. 13B, depicts a karyogram graph wherein the normalized bin scores 1304 of the subset of baseline normalized embryo samples is shown against the non-constant variance of non-normalized bin scores 1308. The HMM can operate in a non-homogenous fashion to accommodate locus specific variability.

There are various other non-HMM methods such as circular binary segmentation, greedy algorithms, and others that can be used to assign copy number states and still remain in the scope of this disclosure.

In various embodiments, the systems and methods disclosed herein have the ability to accurately determine the presence of complex sex aneuploidy in an embryo. The BLUEFUSE® methods discussed above cannot, for example, provide automatic complex sex aneuploidy calls of 47:XXY (sex aneuploidy), 47:XXX (sex aneuploidy), 69:XXY (triploidy) or 69:XYY (triploidy).

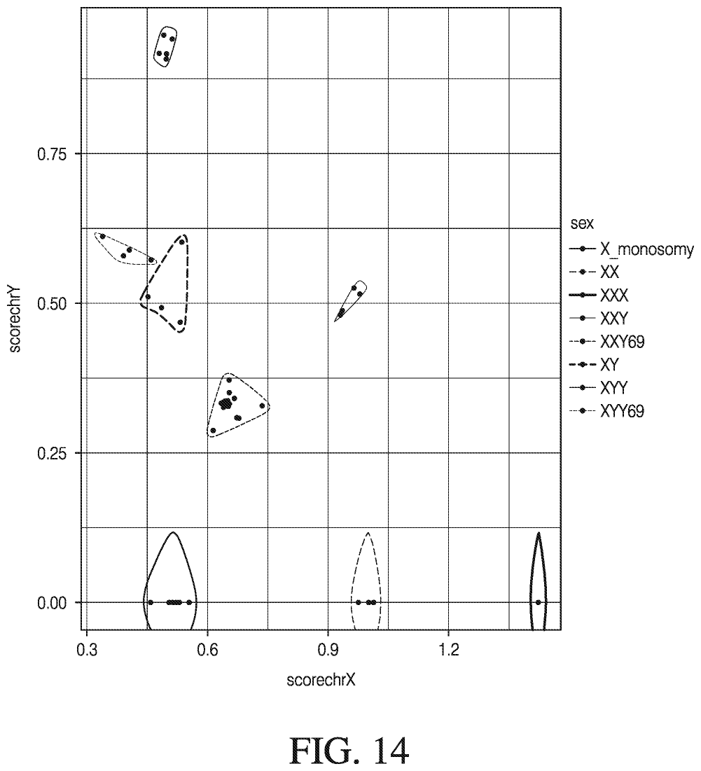

FIG. 14 is a plot that depicts a method that uses chromosomal clusters to determine complex embryo sex aneuploidy, in accordance with various embodiments. This method assigns sex aneuploidy status using a machine learning method such as k nearest neighbors on vectors comprised of: {proportion of sequences aligned to X, bin normalized chromosome X score, proportion of sequences aligned to Y, bin normalized Y score} with a classification method such as k-nearest neighbors with Mahabalonis statistical distance.

In various embodiments, the systems and methods disclosed herein can also utilize neural network methods and other “artificial intelligence” methods. That is, bin scores from across the genome can be processed with neural learning multi-layer perceptron methods to predict aneuploidy status.

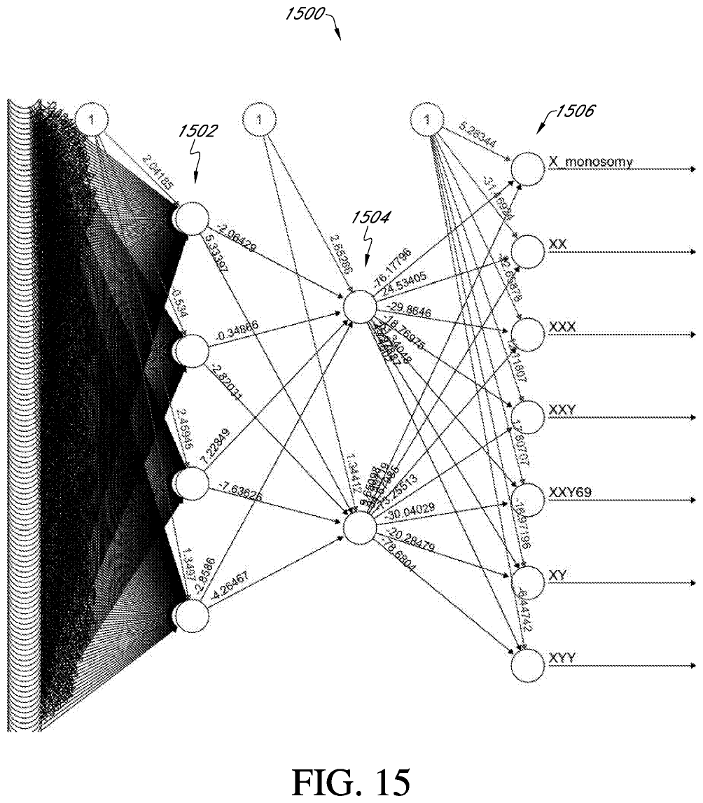

In various embodiments, the neural network topology 1500 used to specify the input of all or some of the bin scores across the genome feeding into feed forward network is comprised of two hidden layers containing four 1502 and two nodes 1504 respectively along with a complex sex aneuploidy outcomes/calls 1506, as shown in FIG. 15. Backpropagation can then be used to construct the neural network weights over a set of training data for which embryo sex aneuploidy status is known.

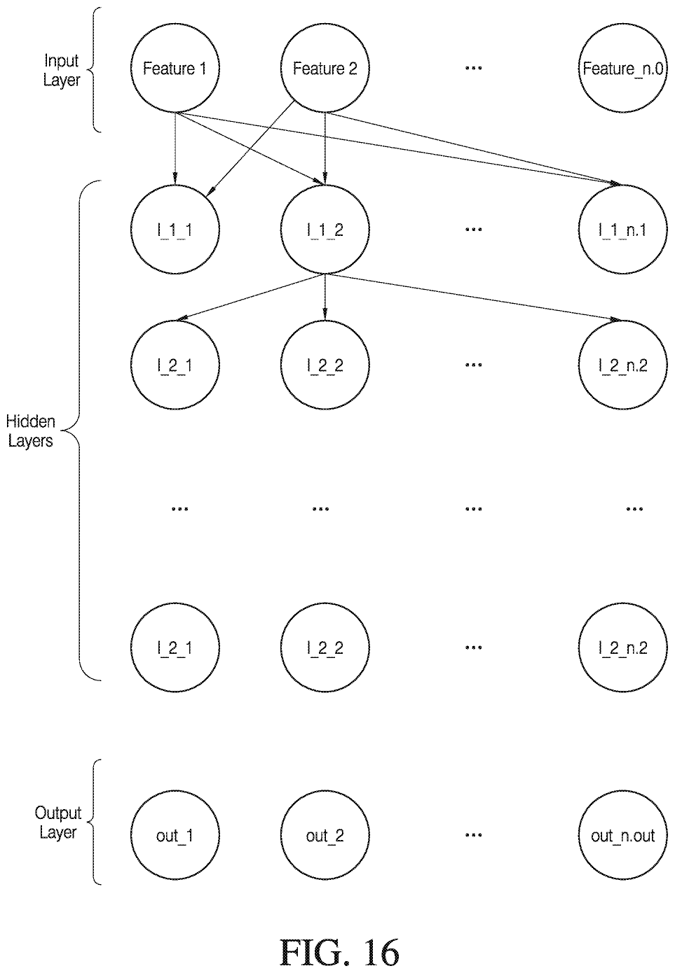

FIG. 16 is a depiction of a feed forward network structure, in accordance with various embodiments. In various embodiments, the input to the network (input layer) is a sub-set of normalized bin scores, as constructed in the “de-noising and normalization” description above or through a similar process, by default, all normalized bins in chromosomes X and Y and all autosome chromosomes (chromosomes 1-22 of the human genome) are used. In various embodiments, a sub-set of chromosomes or chromosome bins may also be used, as determined by inspection or estimated by processes to determine which bins are more important to sex determination.

The hidden layers of a network lie between input and output. In various embodiments, a neural network for identifying complex sex aneuploidy in embryos contains two hidden layers where the first hidden layer is comprised of four nodes, the second hidden layer is comprised of two nodes, and each layer has an additional bias node. It should be appreciated, however, that differing numbers of hidden layers with differing nodes can also be used depending on the requirements of the particular application.

The final output layer has one node for each of the possible outcomes (in this case, one node for each sex state.)

The structure of each non-input node can be a standard perceptron where the output is a nonlinear “activation function” of inputs. By default the activation function can be a rectifier linear unit (ReLU) although ELU, sigmoid, ArcTangent, Step, softmax and many other activation functions can be used in the scope of this disclosure.

With a ReLU activation the output, f, given node inputs, x, is max(0, x).

It should be understood, however, that many other types of neural networks can be applied in the scope of this disclosure; for example, convolutional neral networks (with additional pooling and convolutional layers), recurrent neral networks (where nodes have connections to previous nodes), etc.

One of the distinct advantages of the systems and methods, disclosed herein, is that previously run samples and interpretations can be accumulated to inform future decoding which can help train the systems and methods to be more accurate over time. In various embodiments of the systems and methods disclosed herein, knowledge of features and/or translocations in parental samples can also be incorporated into the learning allowing the detection of small translocations.

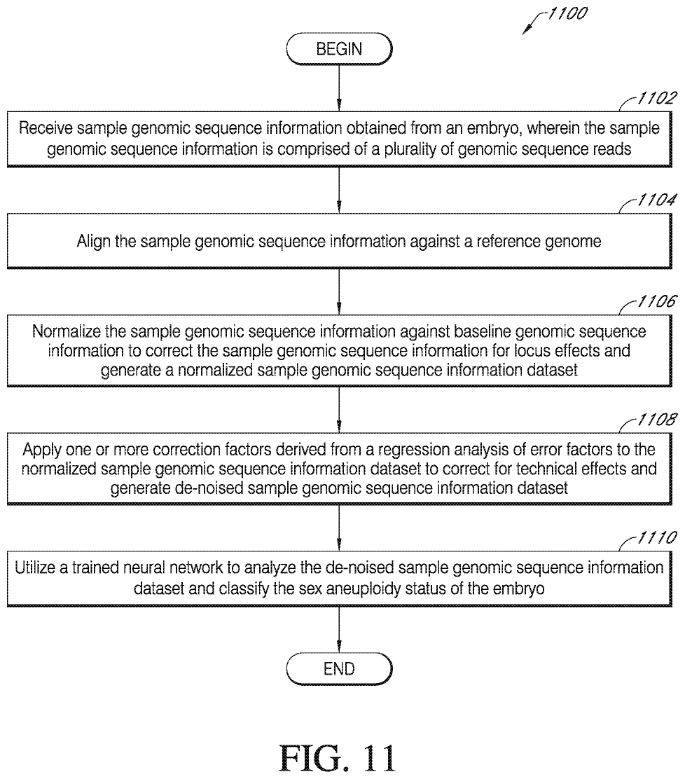

FIG. 11 is an exemplary flowchart showing a method 1100 for identifying sex aneuploidy in an embryo, in accordance with various embodiments.

In step 1102, sample genomic sequence information obtained from an embryo is received. The sample genomic information is comprised of a plurality of genomic sequence reads generated using various genomic sequencing techniques including NGS, PCR, etc. In step 1104, the sample genomic sequence information is aligned against a reference genome. In various embodiments, the reference genome is a human reference genome.

In step 1106, the sample genomic sequence information is normalized against baseline genomic sequence information to correct the sample genomic sequence information for locus effects.

In various embodiments, normalizing the sample genomic sequence information for locus effects involves first setting a bin size. In various embodiments, the bin size is set to 1 megabase (mb). It should be understood, however, that the bin size can be set to any size, including: 100 kb, 500 kb, or any other value between 1 million and to 20 million as long as it doesn't exceed the length of the human genome. Next, the sample genomic sequence information and baseline genomic sequence information is segmented into a plurality of bins based on the selected bin size. Then, the number of genomic sequence reads from the sample genomic sequence information that is aligned to each of the plurality of sample genomic sequence information bins is determined to generate sample bin scores for each of the plurality of sample genomic sequence information bins.

Next, the number of genomic sequence reads from the baseline genomic sequence information that is aligned to each of the plurality of baseline genomic sequence information bins is determined to generate baseline bin scores for each of the plurality of baseline genomic sequence information bins. Then, the sample bin scores are normalized against the baseline bin scores to generate a normalized sample genomic sequence dataset.

In various embodiments, the baseline bin scores were determined by first receiving a plurality of baseline genomic sequence information datasets obtained from euploid embryos. The bin scores for each of the plurality of baseline genomic sequence information datasets were then determined. Next, a subset of baseline genomic sequence information datasets with bin scores that exceed a similarity threshold to the sample genomic sequence information were selected from the plurality of baseline genomic sequence information datasets. Finally, the baseline bin scores were generated by determining the median values of bin scores in the selected subset of baseline genomic information datasets.

In step 1108, one or more correction factors derived from a regression analysis of error factors was applied to correct for technical effects and generate a de-noised sample genomic sequence information dataset.

In step 1110, the de-noised sample sequence information dataset can be analyzed using a trained neural network algorithm/techniques to classify the complex sex aneuploidy status of the embryo.

### Computer-Implemented System

In various embodiments, the methods for identifying chromosomal abnormalities in an embryo can be implemented via computer software or hardware. That is, as depicted in FIG. 9, the methods can be implemented on a computing device/system 904 that includes a Data De-Noising Engine 906, an Artificial Intelligence (AI)/Machine Learning (ML) Powered Interpretation Engine 908 and an AI/ML Powered Sex Aneuploidy Identification Engine 910. In various embodiments, the computing device/system 904 can be communicatively connected to a NGS sequencer 902 and a display device 912 via a direct connection or through an internet connection.

It should be appreciated that the various engines depicted in FIG. 9 can be combined or collapsed into a single engine, component or module, depending on the requirements of the particular application or system architecture. Moreover, in various embodiments, the Data De-Noising Engine 906, an Artificial Intelligence (AI)/Machine Learning (ML) Powered Interpretation Engine 908 and an AI/ML Powered Sex Aneuploidy Identification Engine 910 can comprise additional engines or components as needed by the particular application or system architecture.

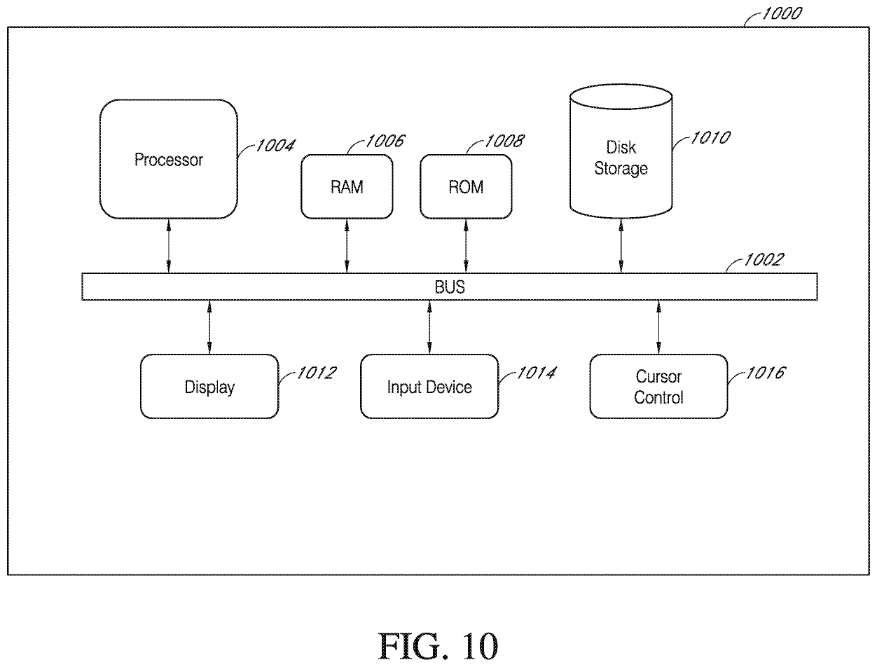

FIG. 10 is a block diagram that illustrates a computer system 1000, upon which embodiments of the present teachings may be implemented. In various embodiments of the present teachings, computer system 1000 can include a bus 1002 or other communication mechanism for communicating information, and a processor 1004 coupled with bus 1002 for processing information. In various embodiments, computer system 1000 can also include a memory, which can be a random access memory (RAM) 1006 or other dynamic storage device, coupled to bus 1002 for determining instructions to be executed by processor 1004. Memory also can be used for storing temporary variables or other intermediate information during execution of instructions to be executed by processor 1004. In various embodiments, computer system 1000 can further include a read only memory (ROM) 1008 or other static storage device coupled to bus 1002 for storing static information and instructions for processor 1004. A storage device 1010, such as a magnetic disk or optical disk, can be provided and coupled to bus 1002 for storing information and instructions.

In various embodiments, computer system 1000 can be coupled via bus 1002 to a display 1012, such as a cathode ray tube (CRT) or liquid crystal display (LCD), for displaying information to a computer user. An input device 1014, including alphanumeric and other keys, can be coupled to bus 1002 for communicating information and command selections to processor 1004. Another type of user input device is a cursor control 1016, such as a mouse, a trackball or cursor direction keys for communicating direction information and command selections to processor 1004 and for controlling cursor movement on display 1012. This input device 1014 typically has two degrees of freedom in two axes, a first axis (i.e., x) and a second axis (i.e., y), that allows the device to specify positions in a plane. However, it should be understood that input devices 1014 allowing for 3 dimensional (x, y and z) cursor movement are also contemplated herein.

Consistent with certain implementations of the present teachings, results can be provided by computer system 1000 in response to processor 1004 executing one or more sequences of one or more instructions contained in memory 1006. Such instructions can be read into memory 1006 from another computer-readable medium or computer-readable storage medium, such as storage device 1010. Execution of the sequences of instructions contained in memory 1006 can cause processor 1004 to perform the processes described herein. Alternatively hard-wired circuitry can be used in place of or in combination with software instructions to implement the present teachings. Thus implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 1004 for execution. Such a medium can take many forms, including but not limited to, non-volatile media, volatile media, and transmission media. Examples of non-volatile media can include, but are not limited to, optical, solid state, magnetic disks, such as storage device 1010. Examples of volatile media can include, but are not limited to, dynamic memory, such as memory 1006. Examples of transmission media can include, but are not limited to, coaxial cables, copper wire, and fiber optics, including the wires that comprise bus 1002.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 1004 of computer system 1000 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein flow charts, diagrams and accompanying disclosure can be implemented using computer system 1000 as a standalone device or on a distributed network of shared computer processing resources such as a cloud computing network

### Experimental Results

The improved systems and methods, disclosed herein, were compared against conventional approaches to identifying chromosomal abnormalities in embryos in order to quantify the improvements in the overall accuracy of the ploidy classifications.

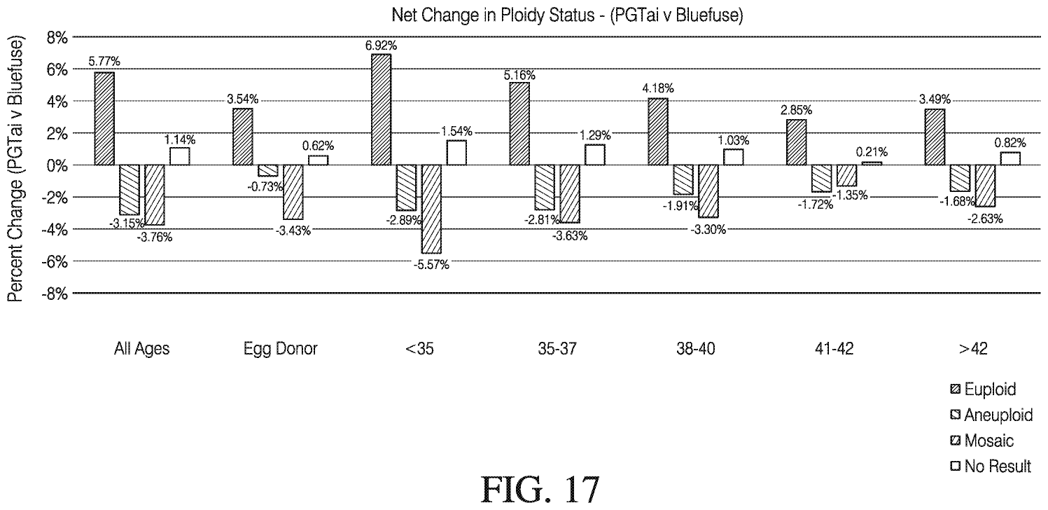

FIG. 17 is a graph showing the net change in the various ploidy classifications when comparing the improved systems and methods disclosed herein (PGTai) against the conventional subjective calling methods (BLUEFUSE® software offered by ILLUMINA®). Over a six-month period, approximately 20,000 embryos were analyzed and classified with the systems and methods described herein (i.e., PGTai). The classification rates were compared to a control population of embryos interpreted by conventional subjective means (i.e., BLUEFUSE®). Classification rates were then assessed by relative comparison, noting overall classification rates achieved by the new systems and methods disclosed herein vs classification rates by conventional means. For example, if the new systems and methods disclosed herein demonstrated that 46% of embryos were classified as euploid, while conventional methodologies indicate that the same source populations produced 41% euploid rates by conventional subjective interpretation, then this is represented as +5%. As described previously, subjective interpretation, especially in the presence of unmitigated noise, is prone to inaccuracies. Specifically, the presence of noise, or an aberrantly low signal-to-noise ratio, results in the over-interpretation. In this setting, over-interpretation is represented by false-positive categorization. In embryo genetics, as one example, this may be represented as true euploids being interpreted as mosaic, or true mosaics being interpreted as aneuploid. As show in FIG. 17, when a sum of approximately 40,000 embryos were analyzed (20,000 by the systems and methods disclosed herein, 20,000 by the conventional subjective methods), material decreases in aneuploid and mosaic rates were observed, while material increase in euploid classification rates were observed. Given the materials were processed in the same laboratories, obtained from the same clinical centers, with only the method of data analysis differing, these results indicated that the improved de-noising processes described herein reduced innacurate calls due to over-interpretation of noise.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1000, whereby processor 1004 would execute the analyses and determinations provided by these engines, subject to instructions provided by any one of, or a combination of, memory components 1006/1008/1010 and user input provided via input device 1014.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

What is claimed is:

1. A method for identifying chromosomal abnormalities in an embryo, comprising:
receiving sample genomic sequence information obtained from an embryo, wherein the sample genomic sequence information is comprised of a plurality of genomic sequence reads;
aligning the sample genomic sequence information against a reference genome;
normalizing the sample genomic sequence information against baseline genomic sequence information to correct the sample genomic sequence information for locus effects and generate a normalized sample genomic sequence information dataset;
applying one or more correction factors derived from a regression analysis of error factors to the normalized sample genomic sequence information dataset to correct for technical effects and generate de-noised sample genomic sequence information dataset; and
identifying copy number variations in the de-noised sample genomic sequence information dataset when a frequency of genomic sequence reads aligned to a chromosomal position on the reference genome deviates from a frequency threshold.

2. The method of claim 1, further including:
generating a karyogram or molecular karyotype from the de-noised sample genomic sequence information dataset.

3. The method of claim 1, wherein normalizing the sample genomic sequence information for locus effects further includes:
setting a bin size;
segmenting the sample genomic sequence information and the baseline genomic sequence information into a plurality of bins based on the bin size;
determining a number of genomic sequence reads from the sample genomic sequence information that is aligned to each of the plurality of sample genomic sequence information bins to generate sample bin scores for each of the plurality of sample genomic sequence information bins;
determining a number of genomic sequence reads from the baseline genomic sequence information that is aligned to each of the plurality of baseline genomic sequence information bins to generate baseline bin scores for each of the plurality of baseline genomic sequence information bins;
normalizing the sample bin scores against the baseline bin scores; and
generating normalized sample genomic sequence information dataset.

4. The method of claim 3, further including:
receiving a plurality of baseline genomic sequence information datasets obtained from euploid embryos;
determining bin scores for each of the plurality of baseline genomic sequence information datasets;
selecting a subset of baseline genomic sequence information datasets, from the plurality of baseline genomic sequence information datasets, with bin scores that exceed a similarity threshold to the sample genomic sequence information; and
generating the baseline bin scores by determining median values of bin scores in the selected subset of baseline genomic sequence information datasets.

5. The method of claim 4, further including:
calculating a similarity value for each of the plurality of baseline genomic sequence information datasets, wherein the similarity value is a measure of how similar each baseline genomic sequence information dataset is to the sample genomic sequence information.

6. The method of claim 4, wherein the similarity value is determined using Eurclidian distance analysis.

7. The method of claim 4, wherein the similarity value is determined using Mahalanobis distance analysis.

8. The method of claim 4, wherein the similarity value is a percent similarity between the baseline genomic sequence information dataset and the sample genomic sequence information.

9. The method of claim 1, wherein the correcting the sample genomic sequence information for sampling effects further includes:
calculating the one or more correction factors using a locally weighted scatterplot smoothing regression analysis.

10. The method of claim 1, wherein the error factor is GC content related.

11. The method of claim 1, wherein the error factor is amplification bias related.

12. The method of claim 1, wherein the error factor is secondary structures related.

13. The method of claim 1, wherein the error factor is nucleosome density related.

14. The method of claim 1, wherein the error factor is miRNA interdiction related.

15. The method of claim 1, wherein the error factor is gene expression related.

16. A system for identifying chromosomal abnormalities in an embryo, comprising:
a data store unit configured to store sample genomic sequence information obtained from an embryo;
a computing device communicatively connected to the data store unit, comprising,
a data de-noising engine configured to receive the sample genomic sequence information from the data store, normalize the sample genomic sequence information against baseline genomic sequence information to correct the sample genomic sequence information for locus effects, and apply one or more correction factors derived from a regression analysis of error factors to correct for technical effects and generate de-noised sample genomic sequence information dataset, and
an interpretation engine configured to identify copy number variations in the de-noised sample genomic sequence information dataset when a frequency of genomic sequence reads aligned to a chromosomal position in the de-noised sample genomic sequence information dataset deviates from a frequency threshold; and
a display communicatively connected to the computing device and configured to display a report containing the identified copy number variations.

17. The system of claim 16, wherein the error factor is GC content related.

18. The system of claim 16, wherein the error factor is amplification bias related.

19. The system of claim 16, wherein the error factor is secondary structures related.

20. The system of claim 16, wherein the error factor is nucleosome density related.

21. The system of claim 16, wherein the error factor is miRNA interdiction related.

22. The system of claim 16, wherein the error factor is gene expression related.

23. The system of claim 16, wherein the computing device further includes:
a sex aneuploidy identification engine configured to utilize a trained neural network to analyze the de-noised sample genomic sequence information dataset to classify the sex aneuploidy status of the embryo.

24. A method for identifying sex aneuploidy in an embryo, comprising:
receiving sample genomic sequence information obtained from an embryo, wherein the sample genomic sequence information is comprised of a plurality of genomic sequence reads;
aligning the sample genomic sequence information against a reference genome;
normalizing the sample genomic sequence information against baseline genomic sequence information to correct the sample genomic sequence information for locus effects and generate normalized sample genomic sequence information dataset;
applying one or more correction factors derived from a regression analysis of error factors to the normalized sample genomic sequence information dataset to correct for technical effects and generate de-noised sample genomic sequence information dataset; and
utilizing a trained neural network to analyze the de-noised sample genomic sequence information dataset and classify the sex aneuploidy status of the embryo.

25. The method of claim 24, further including:
receiving de-noised sample genomic information datasets obtained from a plurality of embryos with known sex aneuploidy classifications; and
updating a neural network with the de-noised sample genomic information datasets to produce the trained neural network.

26. The method of claim 24, wherein the trained neural network is comprised of:
an input layer;
a first hidden layer consisting of four nodes;
a second hidden layer consisting of two nodes; and
an output layer with a plurality of nodes corresponding to different sex aneuploidy classifications.

27. The method of claim 25, wherein the neural network has a feedforward neural network architecture.

28. The method of claim 25, further including applying a back propagation technique to train the neural network.
